(19) United States
(12) Patent Application Publication
(10) Pub. No.: US 2014/0045270 A1
(43) Pub. Date: Feb. 13, 2014

(54) DEVICE HAVING NANOPORE WITH THIOL-CONTAINING MATERIAL ATTACHED TO GOLD LAYER AND METHOD OF ANALYZING NUCLEIC ACID USING THE DEVICE

(71) Applicant: SAMSUNG ELECTRONICS CO., LTD., Suwon-si (KR)

(72) Inventors: Jeo-young SHIM, Yongin-si (KR); Tae-han JEON, Hwaseong-si (KR); Dong-ho LEE, Seongnam-si (KR); Kun-sun EOM, Seoul (KR); Hee-jeong JEONG, Seoul (KR)

(73) Assignee: SAMSUNG ELECTRONICS CO., LTD., Suwon-si (KR)

(21) Appl. No.: 13/773,251

(22) Filed: Feb. 21, 2013

(30) Foreign Application Priority Data
Aug. 9, 2012 (KR) 10-2012-0087351

(51) Int. Cl. G01N 33/50

(52) U.S. Cl. CPC G01N 33/50
USPC 436/94; 422/69; 427/125

(57) ABSTRACT

Provided is a device with a nanopore that has a thiol-containing material bound to a gold layer, methods of producing the devices, and methods of analyzing nucleic acid using the devices.

### CROSS-REFERENCE TO RELATED APPLICATION

This application claims the benefit of Korean Patent Application No. 10-2012-0087351, filed on Aug. 9, 2012, in the Korean Intellectual Property Office, the entire disclosure of which is herein incorporated by reference.

### BACKGROUND

1. Field

The present disclosure relates to devices having a nanopore with an exposed gold layer comprising a thiol-containing material bound to the gold layer, methods of producing the devices, and methods of analyzing nucleic acid using the devices.

2. Description of the Related Art

Methods of analyzing target biomolecules in samples include biopore mimetic systems, which use nanopores or nanogaps. Such systems may be used to detect nucleic acids, for example, by measuring tunneling current or blockade current when DNA or RNA passes through a nanogap. However, in existing systems, the target biomolecules pass through nanopores too quickly, making it was difficult to measure signals caused by the translocation of the target biomolecules.

Accordingly, there remains a need for new devices and methods for analyzing a nucleic acid using nanopores.

### SUMMARY

Provided are devices having nanopores comprising a thiol-containing material bound to a gold layer, methods of producing the devices, and methods of analyzing nucleic acid using the devices.

### BRIEF DESCRIPTION OF THE DRAWINGS

These and/or other aspects will become apparent and more readily appreciated from the following description of the embodiments.

FIG. 1 is sectional views illustrating an example of a method of producing a device comprising a nanopore having an inner wall comprising a first gold layer attached to a thiol-containing material.

FIG. 2 is a sectional view of an example of a device comprising a nanopore having an inner wall comprising a first gold layer attached to a thiol-containing material.

FIG. 3 is an enlarged schematic view of a part of a dotted portion of FIG. 2.

FIG. 4 provides sectional-views illustrating another example of a method of producing a device comprising a nanopore having an inner wall comprising a gold layer attached to a thiol-containing material.

FIG. 5 is a sectional view of an example of a device comprising a nanopore having an inner wall comprising a gold intermediate layer attached to a thiol-containing material.

FIG. 6 is an enlarged schematic view of a left part of a dotted portion of FIG. 5.

FIG. 7 provides sectional-views illustrating an example of a method of producing a device comprising a nanopore having an inner wall comprising a gold layer attached to a thiol-containing material.

FIG. 8 is a sectional view of another example of a device comprising a nanopore having an inner wall comprising a gold intermediate layer attached to a thiol-containing material.

FIG. 9 is an enlarged schematic view of the dotted line of FIG. 8.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

An aspect of the present invention provides a method of producing a nanopore device, the method comprising providing a first substrate layer; contacting the first substrate layer with gold to form a first gold layer; contacting the first gold layer with a first material to form a first material layer; forming a nanopore passing through the layers, such that the first gold layer is exposed on the inner wall of the nanopore; and reacting a thiol-containing material with the first gold layer exposed on the inner wall of the nanopore.

In the respective processes of the method, the contacting processes may be performed by using a known method of attaching a material to a substrate. For example, the contacting processes may be performed by sputtering, spin coating, deposition, or a combination thereof. The deposition may be physical vapor deposition, electrodeposition, or electroless deposition.

The method may include the contacting of gold with a first substrate to form a first gold layer. The gold may be in the form of solid, liquid, or suspension. In addition, the gold may be a composite with other compounds or a compound including gold. The first substrate may be a non-biological derived material. The first substrate may be an insulating material. The first substrate may be formed of a material selected from silicon nitride (Si₃N₄), aluminum oxide (Al₂O₃), silica (SiO₂), a plastic material, such as Teflon™, an elastomer, such as 2-component added cured silicone rubber, glass, and a combination thereof. The first substrate may have a flat shape, for example, a film or membrane shape or an amorphous shape. The “membrane” may include a flat shape material having a predetermined thickness. According to an embodiment of the present invention, at least a portion of the first substrate contacting a first terminal of a nanopore is flat. The substrate may have a layered structure, and for example, a layered structure including a silicon film supported by a support material. A thickness of a substrate having a nanopore may be in a range of about 1 nm to about 1000 nm, for example, about 3.4 nm to about 500 nm or about 1 nm to about 50 nm. The first substrate may be a solid state material.

The method may include the contacting of the first gold layer with a first material to form a first material layer. The first material may include an insulating material. For example, the insulating material may be selected from silicon nitride (Si₃N₄), aluminum oxide (Al₂O₃), silica (SiO₂), a plastic material, such as Teflon™, an elastomer, such as 2-component added cured silicone rubber, glass, and a combination thereof. The first substrate and the first material may each be an insulating material. The first substrate may be silicon nitride and the first material may be aluminum oxide. The first gold layer may have a thickness of 10 nm or less, for example, about 1 to about 10 nm, about 1 to about 9 nm, about 1 to about 7 nm, about 1 to about 5 nm, about 1 to about 3 nm, about 3 to about 9 nm, about 3 to about 7 nm, about 3 to about 5 nm, or about 2 to about 5 nm. A surface of the first substrate on which the gold layer is formed may be flat.

The layers of substrate, gold, material, and/or electrodes form a stack structure. The method may include forming of a nanopore that passes through the stack structure, i.e., through the first substrate, the first gold layer, and the first material layer, in a thickness direction of the stack structure. The thickness direction may be a direction substantially perpendicular to the major surfaces of the layers. In other words, the substrate has a face surface upon which the gold layer and the first material layer are deposited. The thickness direction may be the direction substantially perpendicular to the face surface of the substrate, and substantially parallel to the direction in which the layers are stacked. The forming of the nanopore may be performed by poring. The poring may be performed by, for example, exposure to high-intensity electron beams of field-emission transmission electron microscope (TEM). The nanopore may include a passage through which a fluid flows, and a side surface of the passage may be closed, or at least a portion of the side surface is open to form a gap or passage in a direction substantially perpendicular to the nanopore. The first terminal (end or opening) of the nanopore may be connected to the other terminal thereof, such that the terminals of the nanopore are in fluid communication with each other and connected by a straight (linear) or curved path. The first substrate may be a solid substrate that is capable of supporting the flow of a fluid. A length of a cross-section of the nanopore may be in a range of about 1 nm to about 1000 nm, for example, about 1 nm to about 500 nm, about 1 nm to about 300 nm, about 1 nm to about 200 nm, about 1 nm to about 100 nm, about 1 nm to about 5 nm, about 1 nm to about 10 nm, about 5 nm to about 10 nm or about 1 nm to about 25 nm. The nanopore may have a round or polygonal cross section. When the nanopore has a round cross-section, the length of the cross-section indicates a diameter, and when the nanopore has a polygonal cross-section, the length of the cross-section indicates a shortest distance. The length of the cross-section of the nanopore may be homogenous with respect to a longitudinal direction of the nanopore. The longitudinal direction of the nanopore is not particularly limited. The longitudinal-direction length of the nanopore may be smaller than that of the passing nucleic acid. The longitudinal-direction length of the nanopore may be a distance between a base and another base in a nucleic acid molecule, and may be, for example, from about 3.4 nm to about 500 nm. In addition, the longitudinal-direction length of the nanopore may be a distance between a base and another base in a nucleic acid molecule, and may be, for example 3.4 nm or less. Forming a nanopore that passes, for example perpendicularly, through the stack structure, i.e., through the first substrate, the first gold layer, and the first material layer, exposes the first gold layer on the inner wall of the nanopore. Materials such as including thiol-containing materials may then be reacted with the first gold layer.

The method includes attaching a thiol-containing material to the first gold layer of the nanopore inner wall by reacting the thiol-containing material with the first gold layer of a nanopore inner wall of the stack structure. Thiol reacts with gold (Au) to form a self-assembled monolayer (SAM). The thiol-containing material may be mixed with an organic solvent to prepare a mixed solution, and then a structure including a nanopore may be immersed in the mixed solution, thereby attaching the thiol-containing material to a gold surface. The solvent may be, for example, ethanol. The attachment may be a semi-covalent bond. Due to the reaction, the thiol-containing material may be attached to only the gold layer of the stack structure. Since the thickness of the gold layer is controllable, the thiol-containing material may be attached to only a portion of the nanopore inner wall. The gold layer may be able to withstand harsh chemical washing treatments.

The thiol-containing material may be a material that interacts with a biomolecule. The biomolecule may be selected from a nucleic acid, a protein, a sugar, and a combination thereof. The nucleic acid may be DNA, RNA, PNA, or a combined molecule thereof. The thiol-containing material may be selected from a nucleic acid intercalator, a positively charged material, a conductive material, and a combination thereof. The intercalator may be an aromatic compound having about 10 to about 100 carbon atoms. The intercalator may be selected from naphthalene, anthracene, phenanthrene, pyrene, xlycene, tetracene, or a combination thereof. The intercalator may be a material having about 2 to about 6 benzene rings, and may be, for example, 9-mercaptofluorene, 9-fluorenylmethylthiol, 1-naphthalenethiol, or a combination thereof. The positively charged material may be cysteamine. The conductive material may be, for example, 1,1′,4′,1″-terphenyl-4-thiol. The thiol-containing material may specifically bind to a particular type of nucleotide. For example, the thiol-containing material may specifically bind to adenine nucleotide, thymine nucleotide, guanine nucleotide, or cytosine nucleotide.

The method may further include electrically connecting the first gold layer to a power source, an electric signal measuring device, or a combination thereof. The connection may include an indirect or direct connection. The electric signal measuring device may be used to measure current, voltage, impedance, electric capacitance, or a combination thereof.

The method may further include: contacting a second material with the first gold layer to form a second material layer; contacting gold with the second material layer to form a second gold layer; contacting a third material with the second gold layer to form a third material layer; forming a nanopore passing through the stack structure of the second material layer, the second gold layer, and the third material layer; and attaching a thiol-containing material to the second gold layer exposed on the inner wall of the nanopore by reacting the thiol-containing material with the second gold layer on the inner wall of the nanopore. The respective processes of the method may be performed, unless defined otherwise, in the same or similar manner as described above.

The thiol-containing materials attached to the first gold layer and the second gold layer may be different materials. For example, the thiol-containing materials attached to the first gold layer and the second gold layer may be different materials that bind to different bases.

The method may include forming three or more gold layers by repeatedly performing the additional processes up to the attaching of the thiol-containing material to the second gold layer of the inner wall of the nanopore. For example, the method may include forming of a fourth, fifth, sixth, seventh, or eighth gold layer on the inner wall of the nanopore. The respective gold layers may have attached thiol-containing materials that particularly bind to the respective nucleotide types of a nucleic acid.

Optionally, the contacting of the third material with the formed second gold layer to form the third material layer may be omitted. As a result, the second gold layer may be exposed to a top surface of a device, or other gold layers may be deposited on the second gold layer.

The method may further include the forming of a first chamber that is in fluid communication with the nanopore and is capable of containing a liquid on a first end of the nanopore, and the forming of a second chamber that is in fluid communication with the nanopore and is capable of containing a liquid on the second end of the nanopore.

The method may further include, following the contacting of the first material with the formed first gold layer, contacting an electrode material with the formed first material layer to form a first electrode layer; and contacting a second material with the formed first electrode layer to form the second material layer. In this regard, the nanopore may pass, for example perpendicularly, through the first substrate, the first gold layer, the first material layer, the first electrode layer, and the second material layer. The first material layer and the second material layer may be formed of insulating materials.

The method may further include electrically connecting the first electrode layer to a power source, an electric signal measuring device, or a combination thereof.

Another aspect of the present invention provides a method of producing a nanopore device, wherein the method includes providing a first substrate layer; contacting an electrode material with the first substrate layer to form a first electrode layer; contacting a first material with the first electrode layer to form a first material layer; contacting gold with the first material layer to form a first gold layer; optionally contacting a second material with the first gold layer to form a second material layer; forming a nanopore passing through the stacked structure of the first substrate layer, the first electrode layer, the first material layer, the first gold layer, and the optional second material layer; and reacting a thiol-containing material with the first gold layer exposed on the inner wall of the nanopore.

In the respective processes of the method, the contacting processes may be performed by using a known method of attaching a material to a substrate. For example, the contacting processes may be performed by sputtering, spin coating, deposition, or a combination thereof. The deposition may be physical vapor deposition, electrodeposition, or electroless deposition.

The method may include contacting an electrode material with a first substrate layer to form a first electrode layer. The term “electrode material” refers to a conductive material for use in an electrode. The electrode material may be a metal or carbonaceous material. The metal may be chromium (Cr), copper (Cu), silver (Ag), or a combination thereof. The electrode material may be graphene or carbon nanotube. Graphene or carbon nanotube may be previously manufactured, or may be manufactured in situ. The first substrate may be a non-biological derived material. The first substrate may be an insulating material. The first substrate may be formed of a material selected from silicon nitride (Si₃N₄), aluminum oxide (Al₂O₃), silica (SiO₂), a plastic material, such as Teflon™, an elastomer, such as 2-component added cured silicone rubber, glass, and a combination thereof. The first substrate may have a flat shape, for example, a film or membrane shape or an amorphous shape. The “membrane” may include a flat shape material having a predetermined thickness. According to an embodiment of the present invention, at least a portion of the first substrate contacting a first terminal of a nanopore is flat. The substrate may have a layered structure, and for example, a layered structure including a silicon film supported by a support material. A thickness of a substrate having a nanopore may be in a range of about 1 nm to about 1000 nm, for example, about 3.4 nm to about 500 nm or about 1 nm to about 50 nm. The first substrate may be a solid state material.

The method may include the contacting of the first material with a first substrate to form a first material layer. The first material may include an insulating material. For example, the insulating material may be selected from silicon nitride (Si₃N₄), aluminum oxide (Al₂O₃), silica (SiO₂), a plastic material, such as Teflon™, an elastomer, such as 2-component added cured silicon rubber, glass, and a combination thereof. The first substrate may be formed of an insulating material and the first material may be an insulating material. The first substrate may be formed of silicon nitride and the first material may be aluminum oxide.

The method may include contacting gold with the first material layer to form a first gold layer. The gold may be in the form of a solid, liquid, or suspension. In addition, the gold may be a composite with other compounds or a compound including gold. The first gold layer may have a thickness of 10 nm or less, for example, about 1 to about 10 nm, about 1 to about 9 nm, about 1 to about 7 nm, about 1 to about 5 nm, about 1 to about 3 nm, about 3 to about 9 nm, about 3 to about 7 nm, about 3 to about 5 nm, or about 2 to about 5 nm. A surface of the first substrate on which the gold layer is formed may be flat.

The method may include the contacting of the second material with the formed first gold layer to form the second material layer, which is optional. The second material may include an insulating material. The insulating material may be, for example, silicon nitride, aluminum oxide (Al2O₃), or a combination thereof. The first material and the second material may be identical to or different from each other. For example, the first and second materials may each be aluminum oxide.

The method includes the forming of a nanopore passing, for example perpendicularly, through the stacked structure of the first substrate layer, the first electrode layer, the first material layer, the first gold layer, and the optional second material layer; and the attaching of the thiol-containing material to the first gold layer exposed by the inner wall of the nanopore by reacting the thiol-containing material with the first gold layer exposed on the inner wall of the nanopore. These processes are the same as described above.

The method may further include electrically connecting the first electrode layer to a power source, an electric signal measuring device, or a combination thereof. The connection may include an indirect or direct connection. The electric signal measuring device may be used to measure current, voltage, impedance, electric capacitance, or a combination thereof.

The first gold layer may or may not be connected to a power source, an electric signal measuring device, or a combination thereof.

The method may further include attaching a thiol-containing material to two or more gold layers on the inner wall of the nanopore using processes described above, for example, attaching a thiol-containing material to a second gold layer, a third gold layer, or a fourth gold layer. Accordingly, gold layers exposed on the inner wall of the nanopore may be spaced apart from each other at the same or different intervals along the length of the nanopore, and thiol-containing materials attached to the gold layers may be identical to or different from each other. Gold layers may be separated from each other by an intermediate material, for example, an insulating material. In addition, the thiol-containing materials attached to the respective gold layers may specifically bind to particular types of nucleotides. For example, the thiol-containing materials may specifically bind to an adenine nucleotide, thymine nucleotide, guanine nucleotide, or cytosine nucleotide.

Another aspect of the present invention provides a method of analyzing a nucleic acid in a sample using a nanopore device, the device including: a stack structure of a first substrate layer, a first gold layer, and a first material layer, having a nanopore passing, for example perpendicularly, through the stack structure, wherein a thiol-containing material is attached to the first gold layer exposed on the inner wall of the nanopore; a cis chamber that is in fluid communication with one end of the nanopore and is capable of containing a liquid; and a trans chamber that is in fluid communication with the other end of the nanopore and is capable of containing a liquid, wherein the thiol-containing material is a material that interacts with a nucleic acid, and the first gold layer is electrically connected to a power source and an electric signal measuring device, wherein the method includes: providing a first salt solution as a sample containing a nucleic acid to the cis chamber; providing a second salt solution to the trans chamber; translocating the nucleic acid-containing sample from the cis chamber to the trans chamber; and measuring an electric signal corresponding to the translocation of the nucleic acid-containing sample using an electric signal measuring device connected to the first gold layer.

The method according to the present embodiment includes providing the first salt solution as the sample containing a nucleic acid to the cis chamber. The providing may be distributing the first salt solution to the cis chamber in a manual manner or in a mechanical manner using a mechanical instrument, for example, a pump. The nucleic acid may be selected from DNA, RNA, and a combination thereof. In addition, the nucleic acid may be single-stranded, double-stranded, or a combination thereof. The nucleic acid may have a two-dimensional structure or a three-dimensional structure. The nucleic acid may be isolated. The nucleic acid may be an amplification product.

The term “salt” includes an ionic compound generated from a neutralization reaction between an acid and a base. Such ionic compounds consist of a cation and an anion and are electrically neutral. A solution comprising a dissolved salt, such as NaCl, in water is referred to as an electrolyte, and the electrolyte has electric conductivity. The first salt solution may contain about 1 mM to about 3 M salt. The first salt solution and the second salt solution may contain the same salts or different salts. The salts may be KCl, NaCl, or a combination thereof.

The method according to the present embodiment includes providing the second salt solution to the trans chamber. The providing may be distributing the second salt solution to the trans chamber in a manual manner or in a mechanical manner using a mechanical instrument, for example, a pump. The term “salt” includes an ionic compound generated from a neutralization reaction between an acid and a base. Such ionic compounds each consist of a cation and an anion and are electrically neutral. A solution comprising a dissolved salt, such as NaCl, in water is referred to as an electrolyte, and the electrolyte has electric conductivity. The second salt solution may contain about 0.1 mM to about 0.3 M salt. The salts may be KCl, NaCl, or a combination thereof.

A ratio of a salt concentration of the first salt solution to a salt concentration of the second salt solution may be 10:1 or more, for example, 10-20:1, 10-50:1, 10-100:1, 20-100:1, 50-100:1, or 80-100:1.

The method according to the present embodiment includes translocating the nucleic acid-containing sample from the cis chamber to the trans chamber. The translocating may be performed by applying a driving force to the nucleic acid-containing sample. The translocation may be performed by at least one driving force selected from natural gravity, diffusion, voltage gradient, magnetic gradient, molecular motor, mechanical force, and a combination thereof. According to an embodiment of the present invention, a voltage gradient may be applied between the first end and the other end. In this regard, the first end and the other end contact an electrolytic solution. The electrolytic solution may be, for example, a solution including KCl, NaCl, or a combination thereof.

The method according to the present embodiment includes measuring an electric signal corresponding to the translocation of the nucleic acid-containing sample using the electric signal measuring device connected to the first gold layer. The translocation may be a linear translocation through the nanopore. The electric signal may be caused due to a change in electric characteristics occurring when the nucleic acid linearly moves through the nanopore. The electric characteristics may be current or voltage characteristics. According to an embodiment of the present invention, a decrease or increase in a current amount, and a period of time corresponding thereto, which may occur when the nucleic acid linearly moves through the nanopore, may be measured. That is, a change in electric characteristics over time is measured, and based on the result, the translocation of a corresponding nucleic acid moves is confirmed. The measuring may performed by measuring a current change when the first end and the other end contact an electrolytic solution.

The measuring may be of either a tunneling current between a nucleic acid passing through the nanopore and an electrode or a blockade current due to a nucleic acid passing through the nanopore. The measuring involves measuring an increase in current between a nucleic acid passing through the nanopore and an electrode.

The method may further comprise analyzing a nucleic acid based on the measured electric signal, for example, determining a sequence of a nucleic acid. In addition, the method may further comprise determining the presence or concentration of a nucleic acid sequence based on the measured electric signal.

Another aspect of the present invention provides a method of analyzing a nucleic acid in a sample using a nanopore device, the device including: a stack structure of a first substrate layer, a first electrode layer, a first material layer, a first gold layer, and a second material layer, which is optional, having a nanopore passing, for example, perpendicularly, through the stack structure, wherein a thiol-containing material is attached to the first gold layer exposed on the inner wall of the nanopore; a cis chamber that is in fluid communication with one end of the nanopore and is capable of containing a liquid; and a trans chamber that is in fluid communication with the other end of the nanopore and is capable of containing a liquid, wherein the thiol-containing material is a material that interacts with a nucleic acid, and the first gold layer is not electrically connected to a power source, an electric signal measuring device, or a combination thereof and the first electrode layer is electrically connected to a power source, an electric signal measuring device, or a combination thereof, wherein the method includes: providing a first salt solution as a sample containing a nucleic acid to the cis chamber; providing a second salt solution to the trans chamber; translocating the nucleic acid-containing sample from the cis chamber to the trans chamber; and measuring an electric signal corresponding to the translocation of the nucleic acid-containing sample using an electric signal measuring device connected to the first electrode layer.

The method according to the present embodiment is the same as the method according to the previous embodiment, unless defined otherwise, except that the measuring is performed by measuring an electric signal corresponding to the translocation of the nucleic acid-containing sample using an electric signal measuring device connected to the first electrode layer, not the gold layer.

Regarding the device of the method, the cis chamber is disposed on the side of the second material layer of the stack structure comprising the first substrate layer, the first electrode layer, the first material layer, the first gold layer, and the optional second material layer, and the trans chamber is disposed on the side of the first substrate layer. Accordingly, when a nucleic acid is translocated, the nucleic acid interacts with a thiol-containing material attached to the first gold layer and then, passes by the first electrode layer. As a result, the translocation characteristics of the nucleic acid, for example, a speed thereof, may be controllable due to the thiol-containing material attached to the first gold layer. In the method, the second material layer may be optionally omitted.

Another aspect of the present invention provides a device for analyzing a nucleic acid, the device comprising: a stack structure of a first substrate layer, a first gold layer, and a first material layer, having a nanopore passing, for example, perpendicularly, through the stack structure, wherein a thiol-containing material is attached to the first gold layer exposed on the inner wall of the nanopore; a cis chamber that is in fluid communication with one end of the nanopore and is capable of containing a liquid; and a trans chamber that is in fluid communication with the other end of the nanopore and is capable of containing a liquid.

The stack structure of the device may further include a second material layer disposed on the first gold layer and a second gold layer disposed on the second material layer. A third material may be further disposed on the second gold layer. The second material and the third material may be insulating materials. At least one of the first gold layer and the second gold layer may be connected to an electrode. At least one of the first gold layer and the second gold layer may be connected to a device for measuring electric characteristics. At least one of the first gold layer and the second gold layer may not be connected to an electrode. At least one of the first gold layer and the second gold layer may not be connected to a device for measuring electric characteristics.

Another aspect of the present invention provides a device for analyzing a nucleic acid, the device including: a stack structure of a first substrate layer, a first material layer, and a first gold layer, having a nanopore passing, for example, perpendicularly, through the stack structure, wherein a thiol-containing material is attached to the first gold layer exposed on an inner wall of the nanopore; a cis chamber that is in fluid communication with one end of the nanopore and is capable of containing a liquid; and a trans chamber that is in fluid communication with the other end of the nanopore and is capable of containing a liquid.

The stack structure of the device may further include a second material layer disposed on the first gold layer.

Hereinafter, embodiments of the present invention are described in detail with reference to examples. However, the examples are presented herein for illustrative purpose only, and do not limit the scope of the present invention.

### Example 1

### Production of Nanopore Device Comprising a Gold Layer and Chemical Modification of the Gold Layer

Production of Nanopore Device

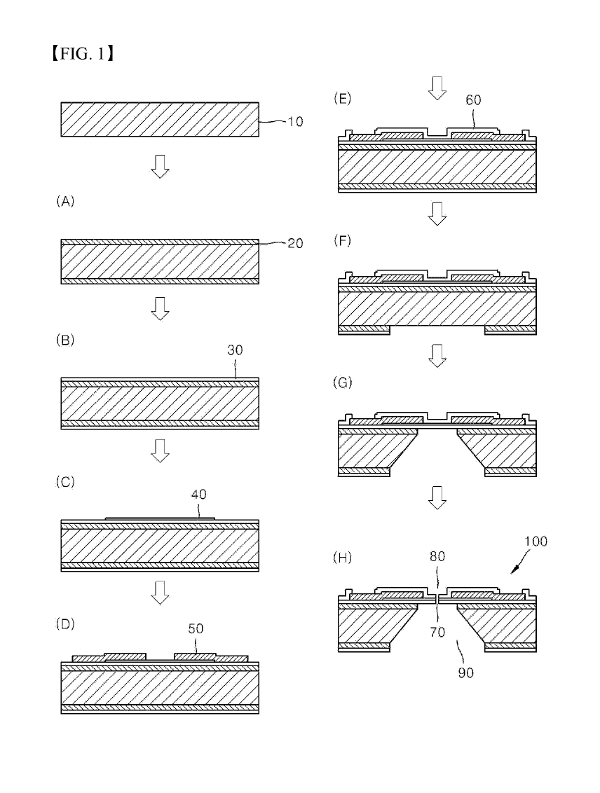

FIG. 1 shows sectional-views illustrating an example of a method of producing a device comprising a nanopore having an inner wall exposing a first gold layer attached to a thiol-containing material.

Referring to FIG. 1, first, SiO₂ is deposited on both surfaces of a Si structure 10 having a thickness (for example, about 300 μm) to form insulating layers each having a thickness (for example, about 300 nm). The deposition may be performed by thermal growth (A).

Then, a low stress SiNx thin film 30 (for example, about 30 nm) for forming a nanopore is formed on each of the insulating layers by low pressure chemical vapor deposition (LPCVD) (B).

Then, an Au/Cr layer 40 that acts as an electrode for a nanogap is formed and then patterned in a nanoribbon shape by E-beam lithography. The nanoribbon is arranged in the form of a pair of electrodes facing each other in the inner space of the nanopore. According to a top view of the nanopore, the electrodes constitute a portion of the nanopore and are spaced apart from each other. In this regard, chromium (Cr) is used to increase an adhesive force of gold with respect to SiNx and located between the gold layer and SiNx layer. The Au/Cr layer 40 included an Au layer having a thickness of 10 nm and a Cr layer having a thickness of 5 nm, and a width thereof was about 50 nm. Then, the Au/Cr layer 40 is entirely cut using an electron beam. In this regard, since Cr binding energy is higher than the binding energy between a silicon nitride and a silicon dioxide, due to the energy quantity required to cut the Cr electrode out, the silicon nitride and the silicon dioxide are also cut, thereby forming a nanopore having a diameter of about 50 nm. Then, silicon nitride and silicon dioxide are regrown on the nanopore to control the size of the nanopore. Regrowth is performed by irradiation of an electron beam with low level of energy on the surrounding of the nanopore. As a result, diameter of the nanopore is controlled to within a few nm. As a result, the Au/Cr layer 40 does not entirely cover the inner wall of the nanopore, and is spaced apart from each other to form a pair of electrodes. Lithography etch process was performed by Lift-off process (C).

Then, an Au/Ti or Au/Cr layer 50 is deposited to form a contact line along which probing or wire-bonding was to be performed using a probe of a probe station, and like the Au/Cr layer 40, the etching is performed by lift-off. The Au/Ti or Au/Cr layer 50 may have a thickness of about 50 nm/10 nm (D).

Then, to insulate metal electrodes, an Al₂O₃ thin film 60 is deposited, and, to obtain a uniform insulating effect, Al₂O₃ is deposited to a thickness of about 30 nm by atomic layer deposition (ALD) (E).

Once the insulating of electrodes is completed, backside etching is performed on the Si substrate to process the nanopore. The SiO₂ layer 20 deposited as an initial insulating layer and the low stress SiNx thin film 30 formed to process the nanopore act as a hardmask (F).

Then, Si on a bottom side of the Si substrate is removed by using a Si etching process using tetramethylammonium hydroxide (TMAH) or KOH. Once buffered oxide etch (BOE) process is performed, the SiO₂ thin film is removed and only the low stress SiNx, Au/Cr (or Au/Ti), and Al₂O₃ insulating layer remains (G). The stack structure of SiN, Au/Cr (or Au/Ti), and the Al₂O₃ insulating layer may have a thickness of about 60 nm. In the Au/Cr (or Au/Ti) layer, Cr or Ti is used to increase an adhesive force of gold with respect to silicon nitride SiNx.

Then, a portion of the Au/Cr layer 40 is removed by using a transmission electron microscope (TEM) to form a nanopore 70 (H). One end of the nanopore 70 is in fluid communication with a cis chamber 80 that is capable of containing a fluid, and the other end of the nanopore 70 is in fluid communication with a trans chamber 90 that is capable of containing a fluid. A cross-section of the nanopore 70 may have a length of about 1 to 10 nm, for example, about 2 nm. Referring to FIG. 1, the cis chamber 80 and the trans chamber 90 are partially not illustrated, and are vessels that are capable of containing a fluid and are not electrically connected to the Au/Ti or Au/Cr 50.

(2) Chemical Modification of the Gold Layer Exposed on the Inner Wall of the Nanopore

A thiol-containing material is reacted with the portion of the gold layer exposed on an inner wall of a nanopore to bind the thiol-containing material to the gold layer. More specifically, a thiol-containing material having a chemical moiety enabling control of a DNA translocation speed is reacted with the formed gold layer and attached thereto. A surface of a nanopore device and contaminants remaining on a surface of the nanopore may first be removed by UV ozone cleaning.

The thiol-containing material, such as an intercalator moiety (e.g., DNA intercalator), a positively charged material, a conducting material, or a combination thereof, is dissolved in a solvent, for example, methylene chloride, chloroform, ethylene acetate, methanol, ethanol, acetonitrile, or a combination thereof. The nanopore device may then be immersed in the solution. Depending on the chemical moiety, the immersing time may be in the range of about 2 hours to about 24 hours.

When the reaction is complete, the nanopore device is taken out of the solution and then washed. The nanopore device is washed three times using the solvent used for the reaction, and then immersed in solvent for 24 hours to completely remove residue of the reaction. Washing may be performed (e.g., three times using a MeOH solution).

The chemical moiety enabling control of DNA translocation speed may be a single moiety or a plurality of different types of moieties. The intercalator moiety may include, for example, 9-mercaptofluorene, 9-fluorenylmethylthiol, 1-naphthalenethiol, or a combination thereof. The positively charged material may comprise a hydrocarbon compound with an amino group at one end and a thiol group at the other end, for instance, cysteamine, 4-amino-1-butanethiol, 5-amino-1-pentanethiol, 6-amino-1-hexanethiol, 7-amino-1-heptanethiol, 8-amino-1-octanethiol, 9-amino-1-nonanethiol, 10-amino-1-decanethiol, 11-amino-1-undecanethiol, 11-amino-1-undecanethiol, or a combination thereof. The hydrocarbon compound may have normal carbon chain, for example, with C1-C20. The conducting material may include thiol group containing-conducting monomer such as phenylene, acetylene, vinylene, thiophene, or a combination thereof, or a polymer containing the same. The conducting material may be a thiophenyl, biphenyl-4-thiol, 1,1′-4′-1″-terphenyl-4-thiol, 4′-mercaptobiphenylcarbonitrile, or a combination thereof, or a polymer containing the same.

An electrode, a power source, or an electric signal measuring device may be connected to a device as described above.

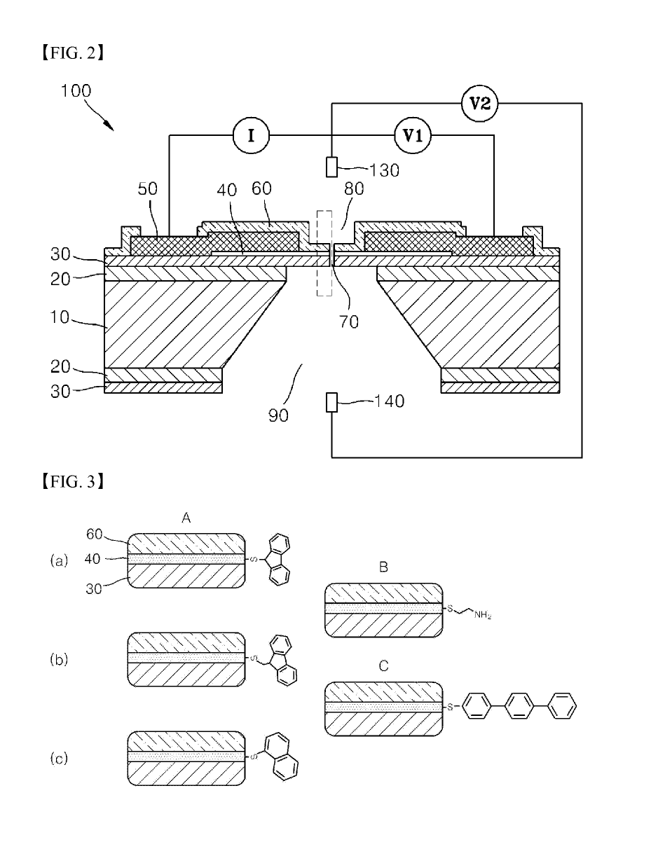

FIG. 2 is a sectional view of an example of a device 100 comprising a nanopore 70 having an inner wall exposing a first gold layer attached to a thiol-containing material.

The device 100 may include: a stack structure of a first substrate 30, a first Au/Cr layer 40, and a first material layer 60 stacked in this stated order having the nanopore 70 passing therethrough in a thickness direction thereof (i.e., generally perpendicular to the largest surfaces of the stacked layers, or generally parallel to the direction in which the layers are stacked), wherein a thiol-containing material is attached to a gold layer exposed on or through an inner wall of the nanopore 70; a cis chamber 80 that is in fluid communication with one end of the nanopore 70 and is capable of containing a liquid; and a trans chamber 90 that is in fluid communication with the other end of the nanopore 70 and is capable of containing a liquid. The first substrate 30 may be a layer of insulating material, for example, a silicon nitride layer. The first material layer 60 may be formed of an insulating material, for example, an aluminum oxide. The first substrate 30 may be supported by a layered structure of SiO₂ 20, silicon 10, and SiO₂ 20. The first Au/Cr layer 40 is connected to another gold layer 50, and the gold layer 50 may be electrically connected to a power source V1, an electric signal measuring device I, or a combination thereof, thereby electrically connecting the first Au/Cr layer 40 to the power source, electrical signal measuring device, or combination thereof. The device 100 may further include a pair of electrodes 130 and 140 that are arranged to apply a voltage through the nanopore 70, and a power source V2 electrically connected to the electrodes 130 and 140. An electric signal measuring device may be electrically connected to the electrodes 130 and 140. The electrodes 130 and 140 may be formed of a material selected from chromium, gold, copper, silver, carbon nanotube, graphene, or a combination thereof. A gold layer of a nanopore may define a portion of the inner wall of the nanopore and a material having thiol may be attached to the gold layer.

FIG. 3 is an enlarged schematic view of a part of a dotted portion of FIG. 2. FIGS. 3A, 3B, and 3C respectively illustrate stack structures of the first substrate layer 30, the first Au/Cr layer 40, and the first material layer 60 which are stacked in this stated order, wherein an intercalator moiety, a positively charged material, and a conducting moiety are attached to a gold layer. In FIG. 3A, (a), (b), and (c) respectively show attachments of 9-mercaptofluorene, 9-fluorenylmethylthiol, and 1-naphthalenethiol. FIGS. 3B and 3C respectively show attachments of cysteamine as a positively charged material and terphenyl thiol as a conductive moiety.

### Example 2

### Production of Nanopore Device Including a Gold Layer and Chemical Modification of the Gold Layer

According to an embodiment of the present invention, in a process of manufacturing a nanopore device according to Example 1, after the process (C) and before the forming of the Au/Cr layer 50 (D), a first material layer 60 and a graphene layer 62 are stacked, and a gold layer 40, which is separated from an electrode, is formed on an inner surface of a nanopore. That is, a thiol-containing material was attached to the gold layer 40, not an electrode, and the gold layer 40, which is not an electrode connected to a power source, an electric signal measuring device, or a combination thereof, was used in chemical modification.

Production of Nanopore Device

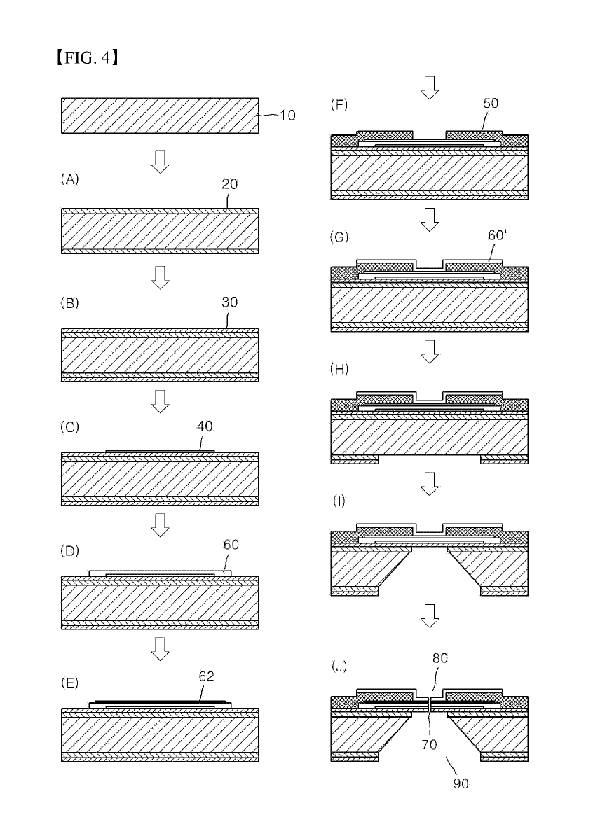

FIG. 4 provides sectional-views illustrating another example of a method of producing a device comprising a nanopore having an inner wall exposing a gold layer attached to a thiol-containing material.

Referring to FIG. 4, first, SiO₂ was deposited on both surfaces of a Si structure 10 having a thickness (for example, about 300 μm) to form insulating layers each having a thickness (for example, about 300 nm). The deposition may be performed by thermal growth (A).

Then, a low stress SiNx thin film 30 (for example, about 30 nm) for forming a nanopore was formed on each of the insulating layers by low pressure chemical vapor deposition (LPCVD) (B).

Then, an Au/Cr layer 40 that is to be used in chemical modification of a nanogap is formed and, then, patterned in a nanoribbon shape by E-beam lithography. The deposited nanoribbon is completely cut by TEM. A diameter of the nanopore is controlled by using a regrowth process. As a result, the nanoribbon does not entirely cover the inner wall of the nanopore, and is spaced apart from each other to form a pair of electrodes.

Since the nanoribbon is used only for chemical modification, the nanoribbon was manufactured in a floating shape not to be connected to other electrodes, such as a graphene electrode or a contact electrode, so as not to act as an electrode. In this regard, chromium (Cr) is used to increase an adhesive force of gold with respect to SiNx. A thickness of the formed Au layer and the Cr layer 40 is about 10 nm and 5 nm, respectively, and a width thereof is about 50 nm. Lithography etch process is performed by Lift-off process (C).

Then, an Al₂O₃ thin film 60 is deposited to insulate a graphene electrode. The deposition was performed by ALD to obtain a uniform thickness of about 30 nm (D).

Then, an electrode process is performed to measure signals. First, a graphene 62 is transferred and then patterned in a nanoribbon shape. The patterning was performed by E-beam lithography. The patterning of graphene was performed by plasma etching (E).

Then, an Au/Ti or Au/Cr layer 50 is deposited to form a contact line along which probing or wire-bonding was to be performed by using a probe of a probe station. The Au/Ti or Au/Cr layer 50 may have a thickness of about 50 nm/10 nm (F).

Then, an Al₂O₃ thin film 60′ is deposited to insulate a metal electrode, and is formed to have a thickness of about 30 nm by ALD to obtain uniform insulating effects (G).

Once the insulating of an electrode is completed, reactive ion etching (RIE) was performed on a bottom surface of the Si substrate 10. The SiO₂ layer 20 deposited as an initial insulating layer and the low stress SiNx thin film 30 formed to process the nanopore act as a hardmask (F). As a result, portions of the SiO₂ layer 20 and the low stress SiNx thin film 30 on the bottom surface of the Si substrate 10 are removed by etching.

Then, a lower portion of the Si substrate 10 is etched by using tetramethylammonium hydroxide (TMAH) or KOH. The SiO₂ thin film 20 is removed by buffered oxide etch (BOE).

As a result, only the low stress SiNx 30, the Au/Cr (or Au/Ti) 40, the graphene 62, and the Al₂O₃ insulating layer 60′ remain (I). The stacked structure of SiNx 30, Au/Cr (or Au/Ti) 40, graphene 62, and Al₂O₃ insulating layer 60′ may have a thickness of about 100 nm. In the Au/Cr (or Cr/Ti) layer 40, Cr or Ti is used to increase an adhesive force of gold with respect to silicon. Selectively, the SiNx 30 is etched to remove at least a portion thereof to expose the Au/Cr (or Au/Ti) 40 of the stacked structure downward.

Then, a portion of the stack structure is removed by using a transmission electron microscope (TEM) to form a nanopore 70 (J). One end of the nanopore 70 is in fluid communication with a cis chamber 80 that is capable of containing a fluid, and the other end of the nanopore 70 is in fluid communication with a trans chamber 90 that is capable of containing a fluid. A cross-section of the nanopore 70 may have a length of about 1 to 10 nm, for example, about 2 nm. Referring to FIG. 4, the cis chamber 80 and the trans chamber 90 are partially not illustrated, and are vessels that are capable of containing a fluid and may not be electrically connected to the graphene 62.

(2) Chemical Modification of a Gold Layer Exposed on the Inner Wall of the Nanopore

A thiol-containing material is reacted to the formed Au/Cr layer 40 exposed by a nanopore to bind the thiol-containing material specifically to the Au/Cr layer 40. In detail, in a nanopore device manufactured by using the method, a thiol-containing material having a chemical moiety enabling control of a DNA translocation speed is reacted with the formed gold layer. First, a surface of a nanopore device and contaminants remaining on a surface of the nanopore are removed by UV ozone cleaning.

As the thiol-containing material, an intercalator moiety, a positively charged material, a conducting material, or a combination thereof, which have thiol, is dissolved in a solvent, for example, methylene chloride, chloroform, ethylene acetate, methanol, ethanol, acetonitrile, or a combination thereof, and then a nanopore device is immersed in the solution. Depending on the chemical moiety, the immersing time is in a range of about 2 hours to about 24 hours.

When the reaction was completed, the nanopore device is taken out of the solution and then washed. The nanopore device is washed three times using the solvent used for the reaction, and then immersed in the solvent for 24 hours to completely remove residue of the reaction. Then, washing is performed three times using a MeOH solution. The chemical moiety may be a single type of moiety or a plurality of types of moieties. The intercalator moiety may include, for example, 9-mercaptofluorene, 9-fluorenylmethylthiol, 1-naphthalenethiol, or a combination thereof. The positively charged material may be cysteamine. The positively charged material may include cysteamine.

A power source or an electric signal measuring device is connected to the graphene electrode 62 of the device manufactured as described above. In addition, the electrodes 130 and 140 are arranged to apply voltage through the nanopore, and a power source (V2) may be electrically connected to the electrodes 130 and 140.

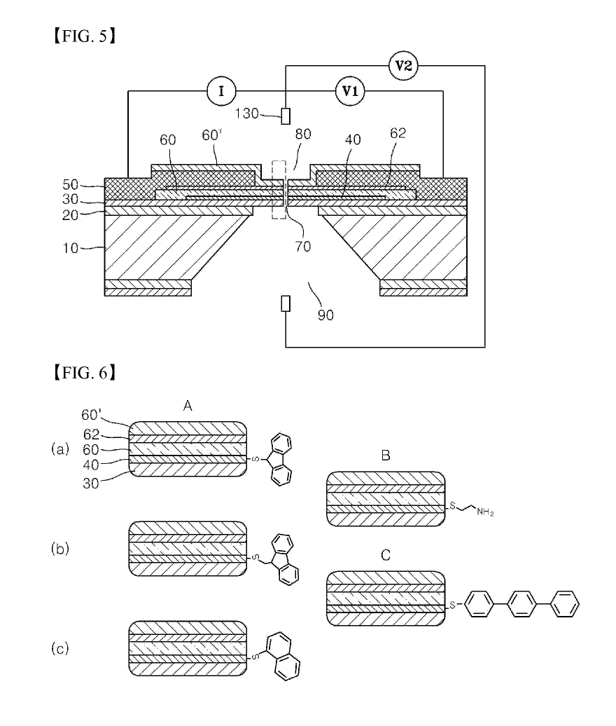

FIG. 5 is a sectional view of an example of a device comprising a nanopore 70 having an inner wall exposing a gold intermediate layer 40 attached to a thiol-containing material.

The device 100 may include: a stack structure of a first substrate layer 30, a gold intermediate layer 40, a first material layer 60, a graphene layer 62, and a second material layer 60′ stacked in this stated order, having the nanopore 70 passing therethrough in a thickness direction thereof, wherein a thiol-containing material is attached to the gold intermediate layer 40 exposed on an inner wall of the nanopore 70; a cis chamber 80 that is in fluid communication with one end of the nanopore 70 and is capable of containing a liquid; and a trans chamber 90 that is in fluid communication with the other end of the nanopore 70 and is capable of containing a liquid. The first substrate layer 30 may be formed of an insulating material, for example, a silicon nitride. The first material layer 60 and the second material layer 60′ may be formed of an insulating material, for example, an aluminum oxide. The first substrate layer 30 may be supported by a layered structure of the first substrate layer 30, the SiO₂ 20, the silicon 10, and the SiO₂ 20. The graphene 62 is connected to a gold layer 50, and the gold layer 50 may be electrically connected to a power source V1, an electric signal measuring device I, or a combination thereof. The device 100 may further include a pair of electrodes 130 and 140 that are arranged to apply a voltage through the nanopore 70, and a power source V2 electrically connected to the electrodes 130 and 140. An electric signal measuring device may be electrically connected to the electrodes 130 and 140. The electrodes 130 and 140 may be formed of a material selected from chromium, gold, copper, silver, carbon nanotube, graphene, or a combination thereof. A gold layer of a nanopore may define a portion of the inner wall of the nanopore and a material having thiol may be attached to the gold layer.

FIG. 6 is an enlarged schematic view of a left part of a dotted portion of FIG. 5. FIGS. 6A, 6B, and 6C respectively illustrate stack structures of the first substrate layer 30, the gold intermediate layer 40, the first material layer 60, the graphene layer 62, and the second material layer 60′ which are stacked in this stated order, wherein an intercalator moiety, a positively charged material, and a conducting moiety are attached to the gold intermediate layer 40. In FIG. 6A, (a), (b), and (c) respectively show attachments of 9-mercaptofluorene, 9-fluorenylmethylthiol, and 1-naphthalenethiol. FIGS. 6B and 6C respectively show attachments of cysteamine as a positively charged material and terphenyl thiol as a conductive moiety.

The devices of FIGS. 5 and 6 are examples, and do not limit the scope of the present invention. For example, at least a portion of the gold intermediate layer 40 disposed under the first substrate layer 30 may be removed to expose the gold intermediate layer 40 downward. Alternatively, instead of the location of the first substrate 30 and the gold intermediate layer 40, that is, the lower part of the stack structure, to be symmetry to the graphene 62, the gold intermediate layer 40 may be disposed on the second material layer 60′, and the first substrate 30 may be disposed on the second material layer 60′. Optionally, a portion of the first substrate 30 disposed on a top surface of the gold intermediate layer 40 may be removed to expose the gold intermediate layer 40 upward. The first substrate 30 may be formed of an insulating material.

### Example 3

### Production of Nanopore Device Comprising a Gold Layer and Chemical Modification of the Gold Layer

The manufacturing process according to the present example is different from that according to Example 2 in that the sequence of the Au/Cr layer forming process and the graphene electrode forming process is switched.

Production of Nanopore Device

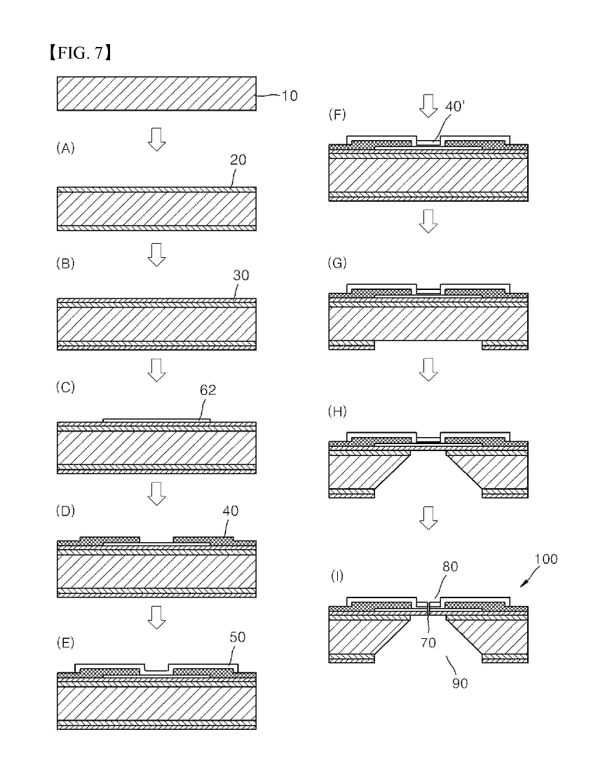

FIG. 7 provides sectional-views illustrating another example of a method of producing a device comprising a nanopore having an inner wall exposing a gold layer attached to a thiol-containing material.

Referring to FIG. 7, first, SiO₂ is deposited on both surfaces of a Si structure 10 having a thickness (for example, about 300 μm) to form insulating layers each having a thickness (for example, about 300 nm). The deposition may be performed by thermal growth (A).

Then, a low stress SiNx thin film 30 (for example, about 30 nm) for forming a nanopore is formed on each of the insulating layers by low pressure chemical vapor deposition (LPCVD) (B).

Then, an electrode process is performed to measure signals. First, a graphene 62 is transferred and then patterned in a nanoribbon shape. The patterning is performed by E-beam lithography. The patterning of graphene was performed by plasma etching (C).

Then, an Au/Ti or Au/Cr layer 50 is deposited to form a contact line along which probing or wire-bonding was to be performed by using a probe of a probe station. In this regard, the etching is performed by lift-off. The Au/Ti or Au/Cr layer 50 may have a thickness of about 50 nm/10 nm (D).

Then, to insulate metal electrodes, an Al₂O₃ thin film 60 is deposited, and to obtain a uniform insulating effect, Al₂O₃ is deposited to a thickness of about 30 nm by atomic layer deposition (ALD) (E).

Then, an Au/Cr layer 40 that is to be used in chemical modification of a nanogap is formed and then patterned in a nanoribbon shape by E-beam lithography. The deposited nanoribbon is completely cut by TEM, and then a diameter of the nanopore is controlled by using a regrowth process. As a result, the nanoribbon does not entirely cover the inner wall of the nanopore, and is spaced apart from each other to form a pair of electrodes. Since the nanoribbon is used only for chemical modification, the nanoribbon is manufactured in a floating shape not to be connected to other electrodes, such as a graphene electrode or a contact electrode, so as not to act as an electrode. In this regard, chromium (Cr) is used to increase an adhesive force of gold with respect to SiNx. A thickness of the formed Au layer and the Cr layer 40 is about 10 nm and 5 nm, respectively, and a width thereof is about 50 nm. Lithography etch process was performed by lift-off process (F).

Optionally, an Al₂O₃ thin film 60′ may be deposited on the Au layer and the Cr layer 40 to insulate the Au layer and the Cr layer 40. The deposition was performed by ALD to obtain a uniform thickness of about 30 nm.

Once a process for a top surface of the Si substrate 10 is completed, reactive ion etching (RIE) was performed on a bottom surface of the Si substrate 10. The SiO₂ layer 20 deposited as an initial insulating layer and the low stress SiNx thin film 30 formed to process the nanopore act as a hardmask (G). As a result, portions of the SiO₂ layer 20 and the low stress SiNx thin film 30 on the bottom surface of the Si substrate 10 were removed by etching.

Then, a lower portion of the Si substrate 10 is etched by using tetramethylammonium hydroxide (TMAH) or KOH. A portion of the SiO₂ thin film 20 is removed by buffered oxide etch (BOE). As a result, only the low stress SiNx 30, the graphene 62, the Al₂O₃ insulating layer 60, and the Au/Cr layer 40 remain (H). As a result, the stack structure of the low stress SiNx 30, the graphene 62, the Al₂O₃ insulating layer 60, and the Au/Cr layer 40 may have a thickness of about 100 nm. In the Au/Cr or Cr/Ti layer 40, Cr or Ti is used to increase an adhesive force of gold with respect to the Al₂O₃ insulating layer 60.

Then, a portion of the stack structure is removed by using a transmission electron microscope (TEM) to form a nanopore 70 (I). One end of the nanopore 70 is in fluid communication with a cis chamber 80 that is capable of containing a fluid, and the other end of the nanopore 70 is in fluid communication with a trans chamber 90 that is capable of containing a fluid. A cross-section of the nanopore 70 may have a length of about 1 to 10 nm, for example, about 2 nm. Referring to FIG. 7, the cis chamber 80 and the trans chamber 90 are partially not illustrated, and are vessels that are capable of containing a fluid and may not be electrically connected to the graphene 62.

(2) Chemical Modification of the Gold Layer Exposed on the Inner Wall of the Nanopore

A thiol-containing material is reacted to the formed Au/Cr layer 40 exposed by a nanopore to bind the thiol-containing material specifically to the Au/Cr layer 40. In detail, in a nanopore device manufactured using the method, a thiol-containing material having a chemical moiety enabling control of a DNA translocation speed is reacted with a formed gold layer. First, the surface of a nanopore device and contaminants remaining on the surface of the nanopore are removed by UV ozone cleaning.

As the thiol-containing material, an intercalator moiety, a positively charged material, a conducting material, or a combination thereof, which have thiol, is dissolved in a solvent, for example, methylene chloride, chloroform, ethylene acetate, methanol, ethanol, acetonitrile, or a combination thereof, and then a nanopore device is immersed in the solution. According to a chemical moiety, the immersing time is controlled to be in a range of about 2 hours to about 24 hours.

When the reaction was completed, the nanopore device is taken out of the solution and then washed. The nanopore device is washed three times using the solvent used for the reaction, and then immersed in the solvent for 24 hours to completely remove residue of the reaction. Then, washing is performed three times using a MeOH solution. The chemical moiety may be a single type of moiety or a plurality of types of moieties. The intercalator moiety may include, for example, 9-mercaptofluorene, 9-fluorenylmethylthiol, 1-naphthalenethiol, or a combination thereof. The positively charged material may be cysteamine. The positively charged material may include cysteamine.

A power source or an electric signal measuring device is connected to the graphene electrode 62 of the device manufactured as described above. In addition, the electrodes 130 and 140 are arranged to apply voltage through the nanopore, and a power source (V2) may be electrically connected to the electrodes 130 and 140.

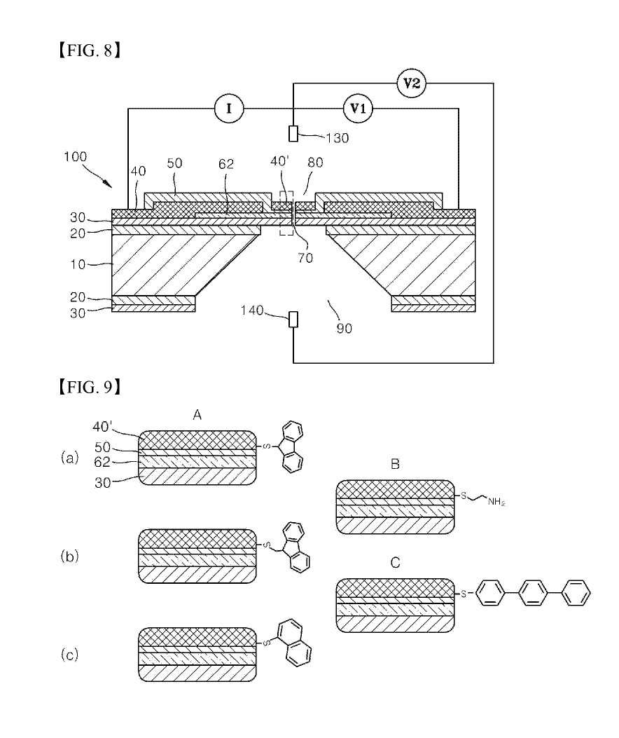

FIG. 8 is a sectional view of another example of a device including a nanopore 70 having an inner wall exposing a gold intermediate layer 40 attached to a thiol-containing material.

The device 100 may include: a stack structure of a first substrate layer 30, a graphene layer 62, a first material layer 50, and a Au/Cr layer 40′ stacked in this stated order, having the nanopore 70 passing, for example, perpendicularly, therethrough, wherein a thiol-containing material is attached to the Au/Cr layer 40′ exposed on an inner wall of the nanopore 70; a cis chamber 80 that is in fluid communication with one end of the nanopore 70 and is capable of containing a liquid; and a trans chamber 90 that is in fluid communication with the other end of the nanopore 70 and is capable of containing a liquid. The first substrate layer 30 may be formed of an insulating material, for example, a silicon nitride. The first material layer 50 and the second material layer 60′ which is optionally disposed on the Au/Cr layer 40′ may be an insulating material (not shown), for example, aluminum oxide. The first substrate layer 30 may be supported by a layered structure of the first substrate layer 30, the SiO₂ 20, and the silicon 10, and the SiO₂ 20. The graphene 62 is connected to a gold layer 40, and the gold layer 40 may be electrically connected to a power source V1, an electric signal measuring device I, or a combination thereof. The device 100 may further include a pair of electrodes 130 and 140 that are arranged to apply a voltage through the nanopore 70, and a power source V2 electrically connected to the electrodes 130 and 140. An electric signal measuring device may be electrically connected to the electrodes 130 and 140. The electrodes 130 and 140 may be formed of a material selected from chromium, gold, copper, silver, carbon nanotube, graphene, or a combination thereof. A gold layer of a nanopore may define a portion of the inner wall of the nanopore and a material having thiol may be attached to the gold layer.

FIG. 9 is an enlarged schematic view of the dotted line of FIG. 8. FIGS. 9A, 9B, and 9C respectively illustrate stack structures of the first substrate 30, the graphene layer 62, the first material layer 50, and the Au/Cr layer 40′ which are stacked in this stated order, wherein an intercalator moiety, a positively charged material, and a conducting moiety are attached to the Au/Cr layer 40′. In FIG. 9A, (a), (b), and (c) respectively show attachments of 9-mercaptofluorene, 9-fluorenylmethylthiol, and 1-naphthalenethiol. FIGS. 9B and 9C respectively show attachments of cysteamine as a positively charged material and terphenyl thiol as a conductive moiety.

The devices of FIGS. 8 and 9 are examples, and do not limit the scope of the present invention. For example, a second material layer may be optionally disposed on the Au/Cr layer 40′. The second material layer may include an insulating material.

It should be understood that the present invention is not limited to the exemplary embodiments described herein. Descriptions of features or aspects within each embodiment should typically be considered interchangeable with other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of producing a nanopore device, the method comprising:
contacting a first substrate with gold and, optionally, an adhesion material, to form a first gold layer;
contacting the first gold layer with a first material to form a first material layer;
forming a nanopore through the first substrate, the first gold layer, and the first material layer in a thickness direction, wherein a portion of the first gold layer is exposed through an inner wall of the nanopore; and
attaching a thiol-containing material to the exposed portion of the first gold layer.

2. The method of claim 1, wherein the first substrate comprises an insulating material and the first material layer comprises an insulating material.

3. The method of claim 1, further comprising:
contacting the first material layer with an electrode material to form a first electrode layer; and
contacting the first electrode layer with a second material to form a second material layer,
wherein the nanopore also passes through the first electrode layer and the second material layer.

4. The method of claim 3, wherein the first material layer and the second material layer each comprise an insulating material.

5. The method of claim 3, further comprising electrically connecting the first electrode layer to a power source, an electric signal measuring device, or a combination thereof.

6. The method of claim 1, wherein the thiol-containing material is a material that interacts with a biomolecule.

7. The method of claim 1, wherein the biomolecule is a nucleic acid, a protein, a sugar, or a combination thereof.

8. The method of claim 1, wherein the thiol-containing material is a nucleic acid intercalator, a positively charged material, a conductive material, or a combination thereof.

9. The method of claim 1, further comprising electrically connecting the first gold layer to a power source, an electric signal measuring device, or a combination thereof.

10. The method of claim 1, further comprising:
contacting the first material layer with gold and, optionally, and adhesion material, to form a second gold layer;
optionally contacting the second gold layer with a third material to form a third material layer,
wherein the nanopore also passes through the second gold layer and the third material layer, and a portion of the second gold layer is exposed through an inner wall of the nanopore; and
attaching a thiol-containing material to the exposed portion of the second gold layer.

11. The method of claim 10, wherein the thiol-containing material attached to the first gold layer and the thiol-containing material attached to the second gold layer are different thiol-containing materials.

12. The method of claim 11, wherein each gold layer is reacted with a different thiol-containing material, and each different thiol-containing material specifically binds to a different nucleic acid.

13. The method of claim 1, further comprising providing a first chamber capable of containing a liquid at one end of the nanopore and providing a second chamber capable of containing a liquid at the other end of the nanopore.

14. A method of producing a nanopore device, the method comprising:
contacting a first substrate with an electrode material to form a first electrode layer;
contacting the first electrode layer with a first material to form a first material layer;
contacting the first material layer with gold and, optionally, an adhesion material, to form a first gold layer;
forming a nanopore passing through the first substrate, the first electrode layer, the first material layer, and the first gold layer in a thickness direction, wherein a portion of the first gold layer is exposed through the inner wall of the nanopore; and
attaching a thiol-containing material to the exposed portion of the first gold layer.

15. The method of claim 14, further comprising contacting the first gold layer with a second material to form a second material layer, wherein the nanopore also passes through the second material layer.

16. The method of claim 14, wherein the electrode material is metal or a carbon-based material.

17. The method of claim 14, further comprising electrically connecting the first electrode layer to a power source, an electric signal measuring device, or a combination thereof.

18. The method of claim 14, further comprising electrically connecting the first gold layer to a power source, an electric signal measuring device, or a combination thereof.

19. A nanopore device comprising
a stack structure comprising
a first substrate layer;
a first gold layer over the first substrate layer; and
a first material layer over the first gold layer;
a nanopore penetrating the stack structure, and each layer thereof, in a thickness direction, wherein a portion of the first gold layer is exposed through an inner wall of the nanopore; and
a thiol-containing material attached to the exposed portion of the first gold layer.

20. The nanopore device of claim 19, further comprising a power source, an electric signal measuring device, or both electrically connected to the first gold layer.

21. The nanopore device of claim 19, further comprising
a cis chamber in fluid communication with one end of the nanopore; and
a trans chamber in fluid communication with the opposite end of the nanopore;
wherein the cis and trans chambers are configured to contain a liquid.

22. A method of analyzing a nucleic acid using a nanopore device of claim 19, the method comprising:
providing a first salt solution comprising a nucleic acid to the cis chamber;
providing a second salt solution to the trans chamber;
translocating the nucleic acid from the cis chamber to the trans chamber; and
measuring an electric signal corresponding to the translocation of the nucleic acid using an electric signal measuring device connected to the first gold layer.

23. A nanopore device comprising
a stack structure comprising
a first substrate;
a first electrode layer over the first substrate;
a first material layer over the first electrode layer; and
a first gold layer over the first material layer;
a nanopore penetrating the stack structure, and each layer thereof, in a thickness direction, wherein a portion of the first gold layer is exposed through an inner wall of the nanopore; and
a thiol-containing material attached to the exposed portion of the first gold layer.

24. The nanopore device of claim 23, further comprising a power source, an electric signal measuring device, or both, electrically connected to the first electrode layer.

25. The nanopore device of claim 24, wherein the first gold layer is not connected to a power source or electric signal measuring device.

26. The nanopore device of claim 23, further comprising
a cis chamber in fluid communication with one end of the nanopore; and
a trans chamber in fluid communication with the opposite end of the nanopore;
wherein the cis and trans chambers are configured to contain a liquid.

27. The device of claim 23, wherein the stack structure further comprises a second material layer on the first gold layer.

28. A method of analyzing a nucleic acid using a nanopore device of claim 23, the method comprising:
providing a first salt solution comprising a nucleic acid to the cis chamber;
providing a second salt solution to the trans chamber;
translocating the nucleic acid sample from the cis chamber to the trans chamber; and
measuring an electric signal corresponding to the translocation of the nucleic acid-containing sample using an electric signal measuring device connected to the first electrode layer.
